(19) United States
(12) Patent Application Publication
(10) Pub. No.: US 2024/0404698 A1
(43) Pub. Date: Dec. 5, 2024

(54) MEDICAL INFORMATION PROCESSING APPARATUS, MEDICAL INFORMATION PROCESSING METHOD, AND RECORDING MEDIUM

(71) Applicant: CANON MEDICAL SYSTEMS CORPORATION, Tochigi (JP)

(72) Inventors: Yuka MATSUMURA, Shioya (JP); Minoru NAKATSUGAWA, Yokohama (JP); Yudai YAMAZAKI, Nasushiobara (JP); Sho SASAKI, Utsunomiya (JP); Longxun PIAO, Nasushiobara (JP)

(73) Assignee: CANON MEDICAL SYSTEMS CORPORATION, Tochigi (JP)

(21) Appl. No.: 18/650,294

(22) Filed: Apr. 30, 2024

(30) Foreign Application Priority Data
May 29, 2023 (JP) 2023-088065

(51) Int. Cl. G16H 50/20; G06F 16/901; G16H 10/60

(52) U.S. Cl. CPC G16H 50/20; G06F 16/9024; G16H 10/60

(57) ABSTRACT

A medical information processing apparatus of the present embodiment is provided with processing circuitry and storage circuitry. The processing circuitry acquires the patient's medical treatment information. The storage circuitry stores a medical treatment ontology related to a medical concept. The processing circuitry identifies an interest medical treatment information group on a medical treatment ontology based on information related to the medical treatment information. The processing circuitry determines a display mode based on the interest medical treatment information group.

### CROSS-REFERENCE TO RELATED APPLICATIONS

This application is based upon and claims the benefit of priority from Japanese Patent Application No. 2023-088065, filed on May 29, 2023; the entire contents of which are incorporated herein by reference.

### FIELD

Embodiments described herein and in the drawings relate generally to a medical information processing apparatus, a medical information processing method, and a recording medium.

### BACKGROUND

As the population ages and with prolonged treatment, the number of patients with multiple comorbidities tends to increase. In particular, cancer treatment may involve concurrent treatment of primary tumors and metastatic sites, or a treatment in combination of surgery, radiation therapy, and chemotherapy. In a hospital, a user who is a physician understands diseases with reference to individual medical treatment information on a screen. However, in a case in which multiple diseases associated with the single medical treatment information are present, other diseases may be overlooked. The user needs to be aware of the progression or signs of the other diseases.

### BRIEF DESCRIPTION OF THE DRAWINGS

FIG. 1 is a diagram illustrating an example of a configuration of a medical treatment information processing system including a medical information processing apparatus according to a first embodiment;

FIG. 2 is a diagram illustrating an example of a configuration of the medical information processing apparatus according to the first embodiment;

FIG. 3 is a flowchart illustrating a procedure of processing executed by the medical information processing apparatus according to the first embodiment;

FIG. 4A is a diagram illustrating the generation of a subgraph structure in the first embodiment;

FIG. 4B is a diagram illustrating the generation of a subgraph structure in the first embodiment;

FIG. 5A is a diagram illustrating the generation of a subgraph structure in the first embodiment;

FIG. 5B is a diagram illustrating the generation of a subgraph structure in the first embodiment;

FIG. 6 is a diagram illustrating identification processing in the first embodiment;

FIG. 7 is a diagram illustrating processing of identifying an interest medical treatment information group as the identification processing in the first embodiment;

FIG. 8 is a diagram illustrating an example of determination processing and display control processing in the first embodiment;

FIG. 9 is a diagram illustrating processing executed by the medical information processing apparatus according to the first embodiment to summarize the processing illustrated in FIGS. 5A, 5B, 7, and 8;

FIG. 10A is a diagram illustrating another example of the determination processing and the display control processing in the first embodiment;

FIG. 10B is a diagram illustrating the other example of the determination processing and the display control processing in the first embodiment;

FIG. 11A is a diagram illustrating yet another example of the determination processing and the display control processing in the first embodiment;

FIG. 11B is a diagram illustrating the yet another example of the determination processing and the display control processing in the first embodiment;

FIG. 12 is a diagram illustrating processing executed by the medical information processing apparatus according to a second embodiment;

FIG. 13 is a diagram illustrating processing executed by the medical information processing apparatus according to a third embodiment; and

FIG. 14 is a diagram illustrating processing executed by the medical information processing apparatus according to a fifth embodiment.

### DETAILED DESCRIPTION

A medical information processing apparatus of the present embodiment is provided with processing circuitry and storage circuitry. The processing circuitry acquires the patient's medical treatment information. The storage circuitry stores a medical treatment ontology related to a medical concept. The processing circuitry identifies an interest medical treatment information group on a medical treatment ontology based on information related to the medical treatment information. The processing circuitry determines a display mode based on the interest medical treatment information group.

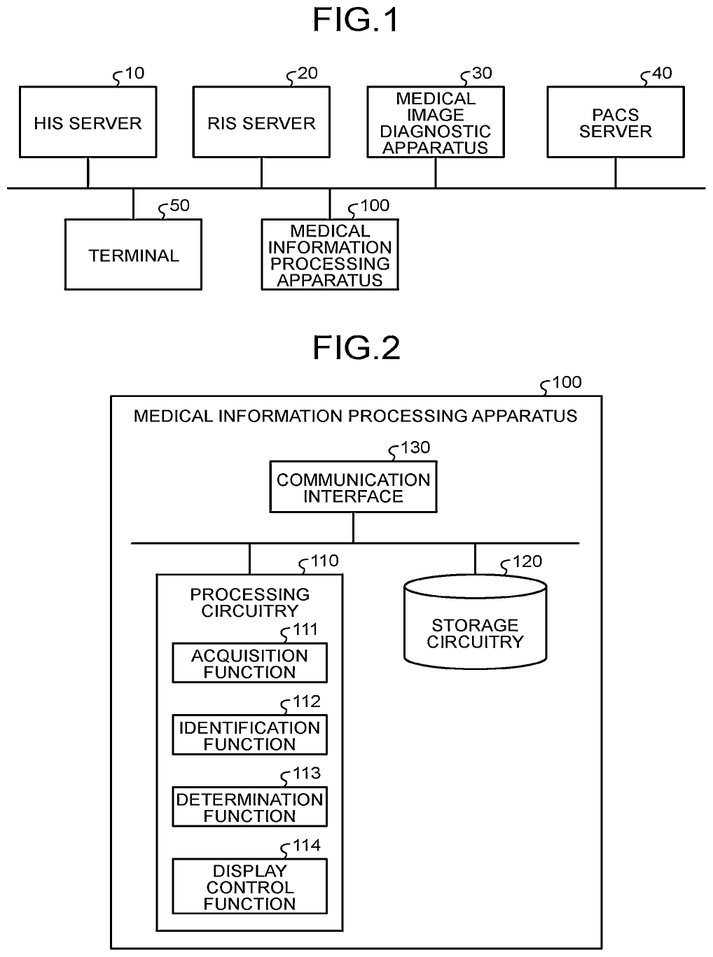

Hereinafter, embodiments of the medical information processing apparatus will be described in detail with reference to the accompanying drawings. Hereinafter, a medical information processing system including the medical information processing apparatus will be described as an example. Although apparatuses are illustrated singly in the medical information processing system illustrated in FIG. 1, the medical information processing system can further include a plurality of the apparatuses in practice.

### First Embodiment

The medical information processing system illustrated in FIG. 1 includes a hospital information system (HIS), a radiology information system (RIS), and a picture archiving and communication system (PACS). The medical information processing system is provided with a HIS server 10, a RIS server 20, a medical image diagnostic apparatus 30, a PACS server 40, a terminal 50, and a medical information processing apparatus 100.

The HIS server 10, the RIS server 20, the medical image diagnostic apparatus 30, the PACS server 40, the terminal 50, and the medical information processing apparatus 100 are connected to, for example, an in-hospital local area network (LAN) installed in a hospital, and transmit information to predetermined devices and receive information transmitted from the predetermined devices. The HIS server 10 may be connected to an external network in addition to the in-hospital LAN.

For example, a user involved in patient care uses the terminal 50. For example, the user is a medical professional such as a physician or a nurse. Examples of the terminal 50 include personal computers (PCs), tablet PCs, personal digital assistants (PDAs), and mobile terminals.

In the HIS, the HIS server 10 illustrated in FIG. 1 manages information generated in the hospital. The information generated in the hospital includes patient information, test order information, and other information.

The patient information includes basic patient information, medical treatment information, and test implementation information. The basic information includes a patient ID, a name, a date of birth, a gender, a blood type, a height, a weight, and other information. The identification information for uniquely identifying the patient is set in the patient ID. The patient's medical treatment information includes information, such as numerical values (measurements) and medical records, as well as information indicating a recording date and time thereof. Examples of the patient's medical treatment information include drug prescriptions by physicians, nursing records by nurses, tests to the laboratory department, and dietary arrangements during hospitalization. For example, the prescriptions are recorded in electronic medical record by the physician, and the nursing records are recorded in electronic medical record by the nurse. The test implementation information includes information on tests conducted in the past and the results of current tests, as well as information indicating a date of the current tests.

Test order information is issued to generate the test implementation information. The test order information includes a test ID, a patient ID, a test code, a medical specialty, a test type, a test site, a scheduled test date and time, and other information. The test ID is an identifier to uniquely identify the test order information. The test code is an identifier to uniquely identify the test. The medical specialty indicates medical specialty classification. The test type indicates a test performed by using medical images. Examples of the test type include an X-ray computed tomography (CT) test, a magnetic resonance imaging (MRI) test, and other tests. The test site includes the brain, kidneys, lungs, liver, and bones.

When the test order information is input by a physician ordering the test, for example, the HIS server 10 transmits the input test order information and patient information identified by the test order information to the RIS. In this case, the HIS server 10 also transmits the patient information to the PACS.

In the RIS, the RIS server 20 illustrated in FIG. 1 manages information pertaining to a radiographic testing service. For example, the RIS server 20 receives the test order information transmitted from the HIS server 10, adds various pieces of setting information to the received test order information, accumulates the information, and manages the accumulated information as test reservation information. Specifically, when receiving the patient information and test order information transmitted from the HIS server 10, the RIS server 20 generates the test reservation information necessary to operate the medical image diagnostic apparatus 30 based on the received patient information and test order information. Information necessary to perform the test, such as the test ID, the patient ID, the test type, and the test site is included in the test reservation information, for example. The RIS server 20 transmits the generated test reservation information to the medical image diagnostic apparatus 30.

The medical image diagnostic apparatus 30 illustrated in FIG. 1 is an apparatus by which a clinical technologist performs a test by imaging a patient, and the like. Examples of the medical image diagnostic apparatus 30 include an ultrasound diagnostic system, an X-ray computed tomography (CT) system, a magnetic resonance imaging (MRI) system, a single photon emission computed tomography (SPECT) system, a positron emission computed tomography (PET) system, a SPECT-CT system in combination of a SPECT system with an X-ray CT system, a PET-CT system in combination of a PET system and an X-ray CT system, and other systems. The medical image diagnostic apparatus 30 is also referred to as a modality device.

The medical image diagnostic apparatus 30 implements a test based on the test reservation information transmitted from the RIS server 20, for example. The medical image diagnostic apparatus 30 then generates information on the implementation of the test and transmits the information to the RIS server 20. In this case, the RIS server 20 receives the test implementation information from the medical image diagnostic apparatus 30 and outputs the received test implementation information to the HIS server 10 as the latest test implementation information. For example, the HIS server 10 receives the latest test implementation information and manages the received test implementation information. The test implementation information includes the test reservation information (such as the test ID, the patient ID, the test type, and the test site), a date and a time for implementing the test, and other information.

In addition, the clinical technologist operates the medical image diagnostic apparatus 30 to image an image of a subject (patient) during the implementation of the test, thereby generating medical image data. Examples of the medical image data include X-ray CT image data, X-ray image data, MRI image data, nuclear medicine image data, ultrasound image data, and other data. The medical image diagnostic apparatus 30 converts the format of the generated medical image data into a format compliant with digital imaging and communication in medicine (DICOM) standards, for example. In other words, the medical image diagnostic apparatus 30 generates medical image data to which DICOM tags are added as metadata. The medical image diagnostic apparatus 30 transmits the generated medical image data to the PACS.

The metadata includes, for example, a patient ID, a test ID, an apparatus ID, an image series ID, conditions related to imaging and the like, and is standardized according to the DICOM standards. The apparatus ID is information to identify the medical image diagnostic apparatus 30. The image series ID is information for identifying a single imaging operation by the medical image diagnostic apparatus 30 and includes, for example, a portion of the subject (patient) that has been imaged, a time of image generation, a slice thickness, and a slice position. For example, a CT scan or MRI scan is performed to obtain tomographic images at each of multiple slice positions as medical image data.

In the PACS, for example, the PACS server 40 illustrated in FIG. 1 receives the patient information transmitted from the HIS server 10 and manages the received patient information. The PACS server 40 has storage circuitry for managing patient information. The PACS server 40 receives the medical image data transmitted from the medical image diagnostic apparatus 30 and stores the received medical image data in association with the patient information in its own storage circuitry, for example. The PACS server 40 reads out the medical image data from its own storage circuitry in response to an acquisition request from the medical information processing apparatus 100 and transmits the data to the medical information processing apparatus 100, for example. The metadata such as the patient ID, the test ID, the apparatus ID, and the image series ID are added to the medical image data stored in the PACS server 40. Therefore, the user can acquire the necessary patient information from the PACS server 40 by performing a search using the patient ID or other information. The user can acquire the necessary medical image data from the PACS server 40 by performing a search using the patient ID, the test ID, the apparatus ID, the image series ID, and other information.

The medical information processing apparatus 100 illustrated in FIG. 1 is a workstation that displays, for example, patient's medical treatment information and images based on medical image data (medical images), as information pertaining to patient care on the display of the terminal 50.

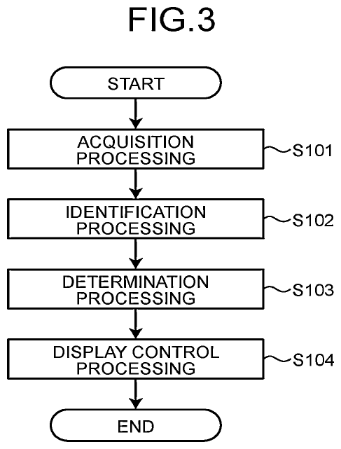

Hereinbelow, the medical information processing apparatus 100 according to the present embodiment will be described in detail. FIG. 2 is a diagram illustrating an example of a configuration of the medical information processing apparatus 100 according to the present embodiment. As illustrated in FIG. 2, the medical information processing apparatus 100 includes processing circuitry 110, storage circuitry 120, and a communication interface 130.

The storage circuitry 120 is connected to the processing circuitry 110 and stores various pieces of information. Specifically, the storage circuitry 120 stores patient information transmitted from each system. For example, the storage circuitry 120 is implemented by a semiconductor memory device such as random access memory (RAM) or flash memory, a hard disk, an optical disk, or the like. Here, the storage circuitry 120 is an example of a storage unit. The communication interface 130 is, for example, a network interface card (NIC) or the like, which communicates with other devices. The storage circuitry 120 is an example of a storage unit.

The processing circuitry 110 controls components of the medical information processing apparatus 100. For example, the processing circuitry 110 executes an acquisition function 111, an identification function 112, a determination function 113, and a display control function 114, as illustrated in FIG. 2. Here, for example, each of processing functions executed by each of the acquisition function 111, the identification function 112, the determination function 113, and the display control function 114, which are components of the processing circuitry 110, is recorded in the storage circuitry 120 in the form of a computer program that can be executed by a computer. The processing circuitry 110 is a processor that reads out each computer program from the storage circuitry 120 and executes the computer program to implement a function corresponding to each program. In other words, the processing circuitry 110 with each computer program read out has each function illustrated in the processing circuitry 110 in FIG. 2. The acquisition function 111, the identification function 112, and the determination function 113 are examples of an acquisition unit, an identification unit, and a determination unit, respectively.

The term “processor” used in the above description means, for example, circuitry such as central processing unit (CPU), graphics processing unit (GPU), or application specific integrated circuit (ASIC). The term “processor” also means circuitry such as a programmable logic device. Examples of the programmable logic device include simple programmable logic device (SPLD) and complex programmable logic device (CPLD). Examples of the programmable logic device also include field programmable gate arrays (FPGA). Provided that the processor is, for example, a CPU, the processor reads out and executes a computer program stored in the storage circuitry 120 to implement a function. On the other hand, provided that the processor is ASIC, for example, the computer program is incorporated directly in the circuitry of the processor, instead of storing the computer program in the storage circuitry 120. Each of the processors in the present embodiment is not limited to a configuration of a single circuit for each processor, but a single processor may also be configured by combining multiple independent circuits to implement functions thereof. Furthermore, a plurality of components in FIG. 2 may be integrated into a single processor to implement the functions thereof.

The overall configuration of the medical information processing system including the medical information processing apparatus 100 according to the present embodiment has been described above. With this configuration, the medical information processing apparatus 100 according to the present embodiment performs the following processing so that the user can understand the relationship between pieces of the medical treatment information. First, in the medical information processing apparatus 100 according to the present embodiment, the acquisition function 111 acquires the patient's medical treatment information. The storage circuitry 120 stores medical ontologies related to medical concepts. The identification function 112 identifies an interest medical treatment information group on a medical treatment ontology based on information related to the medical treatment information. The determination function 113 determines a display mode based on the interest medical treatment information group. The medical treatment information includes information managed by the HIS server 10 and the PACS server 40.

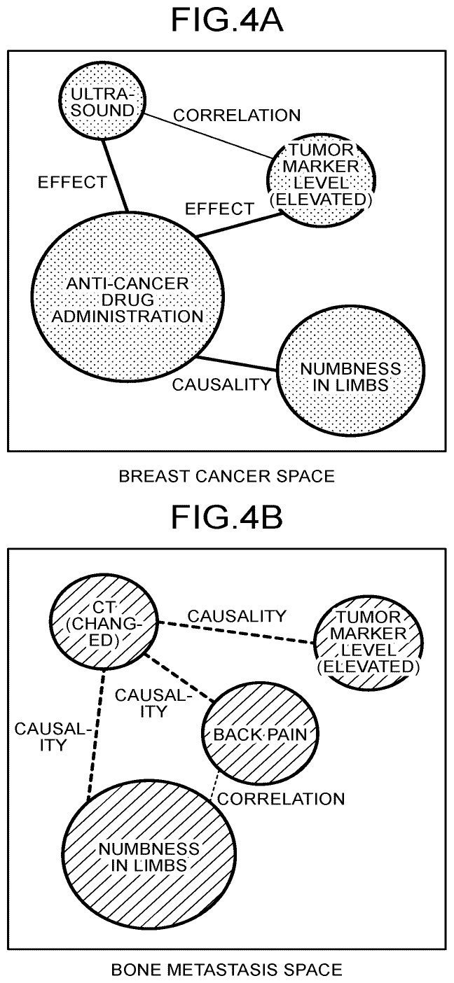

First, processing executed by the medical information processing apparatus 100 according to the present embodiment will be described. FIG. 3 is a flowchart illustrating a procedure of processing executed by the medical information processing apparatus 100 according to the present embodiment.

At step S101 in FIG. 3, calling a computer program corresponding to the acquisition function 111 from the storage circuitry 120 is executed by the processing circuitry 110. In acquisition processing at the step S101, the acquisition function 111 acquires the patient's medical treatment information from the HIS server 10.

At step S102 in FIG. 3, calling a computer program corresponding to the identification function 112 from the storage circuitry 120 is executed by the processing circuitry 110. In identification processing at the step S102, the identification function 112 identifies an interest medical treatment information group on a medical treatment ontology related to the medical concept stored in the storage circuitry 120 based on the information related to the medical treatment information acquired at the step S101. Details of the identification processing are described later.

At step S103 in FIG. 3, calling a computer program corresponding to the determination function 113 from the storage circuitry 120 is executed by the processing circuitry 110. In determination processing at the step S103, the determination function 113 determines a display mode based on the interest medical treatment information group. Details of the determination processing are described later.

At step S104 in FIG. 3, calling a computer program corresponding to the display control function 114 from the storage circuitry 120 is executed by the processing circuitry 110. In display control processing at the step S104, the display control function 114 causes the terminal 50 used by the user to display the display mode determined at the step S103 on its display. Details of the display control processing are described later.

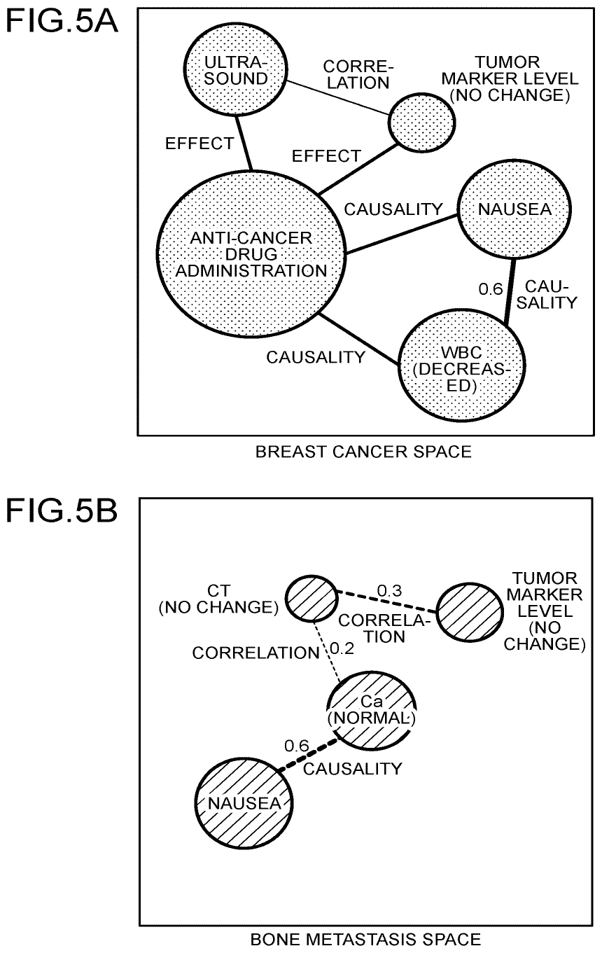

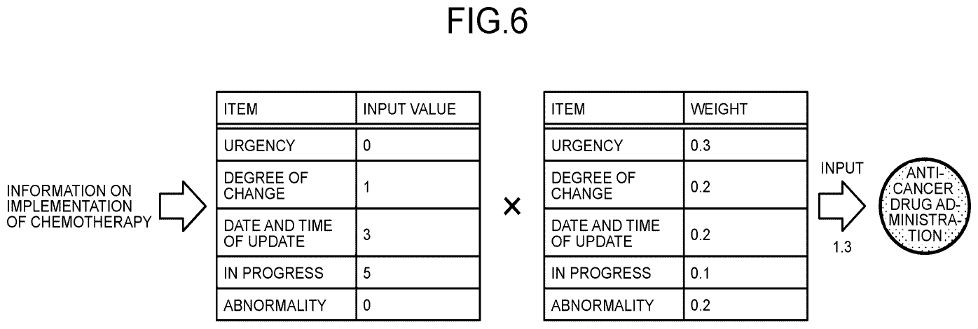

Here, the medical treatment ontology related to the medical concept is generated in advance and stored in the storage circuitry 120. The storage circuitry 120 stores a plurality of the medical ontologies related to a plurality of the medical concepts. For example, a medical treatment ontology is a graph structure in which a medical concept functions as a node, and the relationship between medical concepts functions as an edge. The medical concept indicates a concept related to a disease or a treatment method. FIGS. 4A, 4B, 5A, and 5B are diagrams illustrating the generation of subgraph structures in the present embodiment.

Nodes and edges of a graph structure are determined based on the characteristics of the disease or the treatment method as the medical concepts. For example, the nodes and edges are determined by leveraging machine learning such as rule-based machine learning, graph neural network (GNN) machine learning, or other machine learning models determined based on the characteristics of the disease or the treatment method. Given that the medical concept is present in the plural, the relationship between different medical concepts is established based on the characteristics of each medical concept.

For example, the subgraph structure “breast cancer space” is generated in advance, as illustrated in FIG. 4A. In the subgraph structure “breast cancer space”, the items “anti-cancer drug administration”, “ultrasound”, “tumor marker level (elevated)”, and “numbness in limbs” are determined as nodes. The “effect” of the anti-cancer drug administration, as confirmed by ultrasound, is determined as an edge between the node “anti-cancer drug administration” and the node “ultrasound”. The “effect” of the anti-cancer drug administration confirmed by the tumor marker is determined as an edge between the node “anti-cancer drug administration” and the node “tumor marker level (elevated)”. The “causality”, cause and effect between the anti-cancer drug administration and the numbness in the limb, is determined as an edge between the node “anti-cancer drug administration” and the node “numbness in limbs”. Regarding the breast cancer, the “correlation” indicating to perform an ultrasound due to elevated tumor marker levels is determined as an edge between the node “tumor marker level (elevated)” and the node “ultrasound”.

For example, the subgraph structure “bone metastasis space” is generated in advance as illustrated in FIG. 4B. In the subgraph structure “bone metastasis space”, the items “tumor marker level (elevated)”, “numbness in limbs”, “back pain”, and “CT (changed)” are determined as nodes. The “causality” indicating to perform a CT scan due to elevated tumor marker levels is determined as an edge between the node “tumor marker level (elevated)” and the node “CT (changed)”. The “causality” indicating to perform a CT scan due to numbness in limbs is determined as an edge between the node “numbness in limbs” and the node “CT (changed)”. The “causality” indicating to perform a CT scan due to back pain is determined as an edge between the node “back pain” and the node “CT (changed)”. Regarding bone metastasis, the “correlation” indicating that numbness in limbs and back pain occur is determined as an edge between the node “numbness in limbs” and the node “back pain”.

In the examples illustrated in FIGS. 4A and 4B, the relationships with other nodes vary depending on the disease concepts even in the same medical treatment information. Here, in the examples illustrated in FIGS. 4A and 4B, the individual subgraph structures are separately generated into the “breast cancer space” and the “bone metastasis space” for each disease concept. However, a subgraph structure in which the “breast cancer space” and the “bone metastasis space” are combined may be generated in advance. In the examples illustrated in FIGS. 4A and 4B, although the treatment methods such as the anti-cancer drug administration and the CT scan, elevated tumor marker levels and the like are also exemplified as nodes, information such as treatment methods other than the above-described treatment methods, high values in test results, positive test results, and changes in test results may be determined as nodes. In the examples illustrated in FIGS. 4A and 4B, although the effect, causality, and correlation are also exemplified as the edges, information such as symptoms, complications, and risk may be determined as edges.

Here, weights are determined between edges based on disease characteristics. For example, in the “breast cancer space”, nausea is more causally related to a decrease in white blood cells (WBC; the number of white blood cells), while in the “bone metastasis space”, nausea is more causally related to a decrease in calcium (Ca). The weights between the edges may be determined based on manuals with guidelines, papers and the like, or weights across all the edges may be normalized by using treatment superiority evaluations, research results and comparative results regarding risk factors and the like. The weights between the edges may be determined by leveraging machine learning. The machine learning can be re-trained to fit an individual patient.

For example, in the subgraph structure “breast cancer space” illustrated in FIG. 5A, the items “anti-cancer drug administration”, “ultrasound”, “tumor marker level (no change)”, “WBC (decreased)”, and “nausea” are determined as nodes. The edge “effect” is determined between the node “anti-cancer drug administration” and the node “ultrasound”, the edge “effect” is determined between the node “anti-cancer drug administration” and the node “tumor marker level (no change)”, and the edge “correlation” is determined between the node “tumor marker level (no change)” and the node “ultrasound”. The edge “causality” is determined between the node “anti-cancer drug administration” and the node “WBC (decreased)”, the edge “causality” is determined between the node “anti-cancer drug administration” and the node “nausea”, and the edge “causality” is determined between the node “WBC (decreased)” and the node “nausea”. In the example illustrated in FIG. 5A, a weight of “0.6” is determined at the edge “causality” between node “WBC (decreased)” and the node “nausea”.

For example, in the subgraph structure “bone metastasis space” illustrated in FIG. 5B, the items “nausea”, “Ca (normal)”, “CT (no change)” and “tumor marker level (no change)” are determined as nodes. The edge “causality” is determined between the node “nausea” and the node “Ca (normal)”, the edge “correlation” is determined between the node “Ca (normal)” and the node “CT (no change)”, and the edge “correlation” is determined between the node “CT (no change)” and the node “tumor marker level (no change)”. In the example illustrated in FIG. 5B, a weight of “0.6” is determined at the edge “causality” between the node “nausea” and the node “Ca (normal)”, a weight of “0.2” is determined at the edge “correlation” between the node “Ca (normal)” and the node “CT (no change)”, and a weight of “0.3” is determined at the edge “correlation” between the node “CT (no change)” and the node “tumor marker level (no change)”.

Next, the processing (identification processing) executed by the identification function 112 will be described. The identification function 112 identifies an interest medical treatment information group on the medical treatment ontology related to the medical concept stored in the storage circuitry 120 based on the information related to the medical treatment information acquired by the acquisition function 111.

First, the identification function 112 calculates an input value of each node corresponding to the medical treatment information when the medical treatment information is acquired by the acquisition function 111. Here, the identification function 112 calculates the input value of each node based on the patient's current medical condition by using items such as “urgency”, “degree of change”, “date and time of update”, “in progress”, and “abnormality”. As an example, it will be described that the medical treatment information acquired by the acquisition function 111 includes information on the implementation of chemotherapy, and the identification function 112 calculates the input value for the node “anti-cancer drug administration” corresponding to the information on the implementation of chemotherapy by using each item.

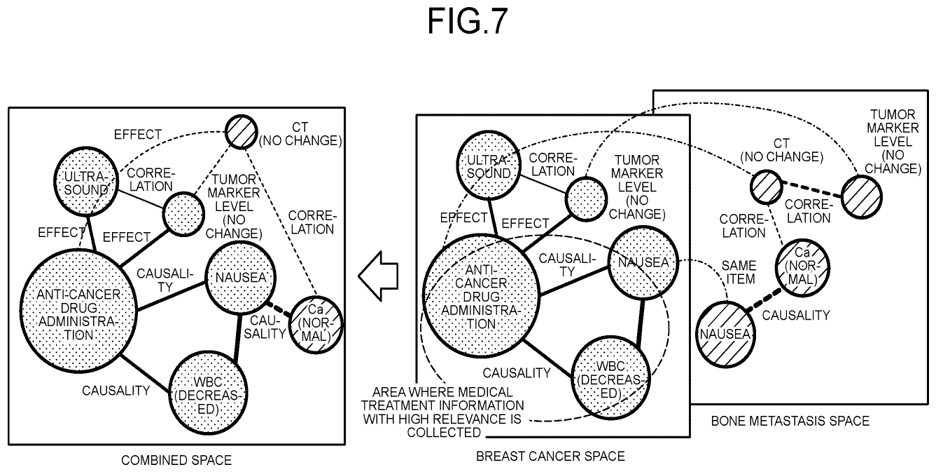

FIG. 6 is a diagram illustrating identification processing in the present embodiment. For example, input values of “0”, “1”, “3”, “5”, and “0” are respectively assigned to the items “urgency”, “degree of change”, “date and time of update”, “in progress”, and “abnormality” of the information on the implementation of chemotherapy. Specifically, the item “urgency” is assigned an input value of “0” because the chemotherapy was implemented expectedly upon the information on the implementation of chemotherapy. In a case in which the degree of change in the patient to which the chemotherapy has been implemented is low upon the information on the implementation of chemotherapy, a low input value of “1” is assigned to the item “degree of change”. In a case in which the date and time of the implementation of the chemotherapy is a new date and time upon the information on the implementation of chemotherapy, a high input value of “3” is assigned to the item “date and time of update”, for example. In a case in which the chemotherapy is being implemented upon the information on the implementation of chemotherapy, a high input value of “5” is assigned to the item “in progress”, for example. In a case in which the abnormality of the result in the chemotherapy is low upon the information on the implementation of chemotherapy, an input value of “0” is assigned to the item “abnormality”.

Weights of “0.3”, “0.2”, “0.2”, “0.1”, and “0.2” are respectively assigned to the items “urgency”, “degree of change”, “date and time of update”, “in progress”, and “abnormality” of the node “anti-cancer drug administration”. In this case, the identification function 112 calculates input values of the node “anti-cancer drug administration” corresponding to the information on the implementation of chemotherapy by 0×0.3+1×0.2+3×0.2+5×0.1+0×0.2=1.3. Here, although each item is added up in the calculation of the input values, each item may be evaluated individually, or urgency may be given top priority.

Next, for each node corresponding to the medical treatment information, the identification function 112 calculates, by the following equation, an internal state hi of a node i from an input value of the node i and an input value of an adjacent node j that is adjacent to the node i according to a graph neural network algorithm, for example.

Here, n indicates the number of adjacent nodes, wᵢⱼ indicates a weight of an edge between the node i and the adjacent node j, hⱼ indicates the internal state of the adjacent node j, wᵢ indicates a weight of the node i, and xᵢ indicates an input value of the node i.

The size of the internal state of the node is reflected in FIGS. 4A, 4B, 5A, and 5B; as the internal state of the node is larger, the size of the circle representing the node is represented to be larger.

The identification function 112 derives a relevance between individual nodes corresponding to the medical treatment information in the graph structure, and identifies a subgraph structure including a node with high relevance and a node adjacent to the node with high relevance as the interest medical treatment information group. Specifically, the identification function 112 extracts, as an axis of medical care to be displayed, a subgraph structure that is an area where medical treatment information with high relevance is collected and in which the overall internal state of a node is high. Here, a plurality of axes of medical care may be extracted as the axis of medical care.

The identification function 112 first evaluates the subgraph structure using the energy function E illustrated in the following equation.

Here, n indicates the number of nodes in the subgraph structure, wᵢⱼ indicates a weight of an edge between the node i and the adjacent node j, hi indicates an internal state of the node i, and hⱼ indicates an internal state of the adjacent node j. The subgraph structure may be evaluated by convolution filtering or pooling filtering.

Next, the identification function 112 extracts an area including the nodes with the high internal states as a subgraph structure. For example, the identification function 112 extracts an area including higher-level nodes with the high internal states or nodes connecting the higher-level nodes as a subgraph structure.

The extracted subgraph structure may be modified according to an attribute of a medical department or medical provider. For example, the extracted subgraph structure may be intentionally modified by adding an internal state to a node in which an attribute of medical treatment information matches the attribute of the medical department or medical provider, and other modifications. For example, a subgraph structure to be extracted may be modified by performing the modification on a node of medical treatment information generated by the breast surgery department to increase an internal state of the node when referenced by a breast surgeon, and other modifications.

Therefore, the identification function 112 combines the medical ontologies based on the medical treatment information to generate a combined medical treatment ontology, and identifies the interest medical treatment information group on the combined medical treatment ontology. The identification function 112 is an example of a combining unit.

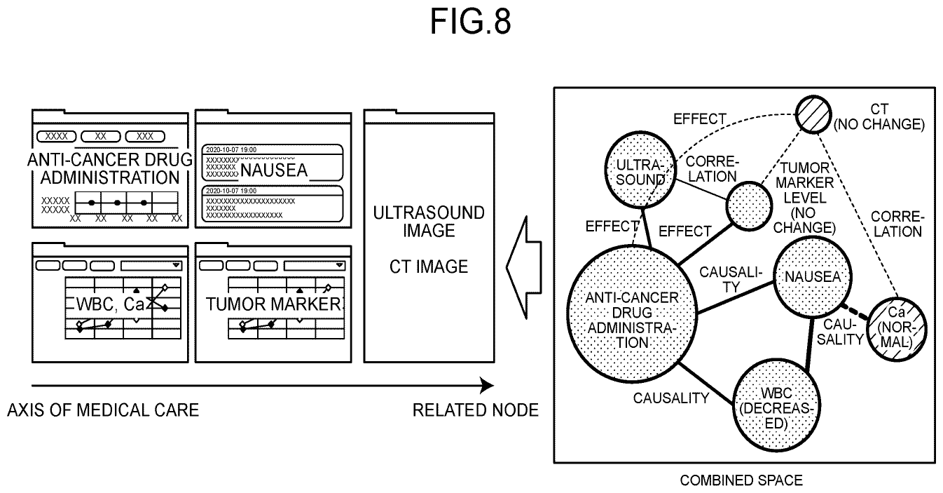

FIG. 7 is a diagram illustrating processing of identifying an interest medical treatment information group as the identification processing in the present embodiment. FIG. 7 illustrates processing of generating a combined medical treatment ontology in which the subgraph structure “breast cancer space” illustrated in FIG. 5A is combined with the subgraph structure “bone metastasis space” illustrated in FIG. 5B. In FIG. 7, for example, the node “nausea” in the subgraph structure “breast cancer space” and the node “nausea” in the subgraph structure “bone metastasis space” are the same item. In FIG. 7, for example, it is assumed that the node “anti-cancer drug administration” in the subgraph structure “breast cancer space” and the node “CT (no change)” in the subgraph structure “bone metastasis space” have the same effect. In this case, in FIG. 7, an area where medical treatment information with high relevance is collected includes the nodes “anti-cancer drug administration”, “WBC (decreased)”, and “nausea” in the subgraph structure “breast cancer space”. The node with high relevance to the subgraph structure “breast cancer space” is the node “Ca (normal)” adjacent to the same node “nausea” in the subgraph structure “bone metastasis space” as the node “nausea” in the subgraph structure “breast cancer space”. The node with high relevance to the subgraph structure “breast cancer space” is the node “CT (no change)” in the subgraph structure “bone metastasis space”, which has the same effect as the node “anti-cancer drug administration” in the subgraph structure “breast cancer space”. In this case, the identification function 112 generates, as a combined medical treatment ontology, a combined space where the edge “effect” is given to between the node “anti-cancer drug administration” in the subgraph structure “breast cancer space” and the node “CT (no change)” in the subgraph structure “bone metastasis space”, and the edge “causality” is given to between the node “nausea” in the subgraph structure “breast cancer space” and the node “Ca (normal)” in the subgraph structure “bone metastasis space”. The identification function 112 then identifies an interest medical treatment information group (anti-cancer drug administration, ultrasound, tumor marker, . . . ) on the combined medical treatment ontology.

Next, processing executed by the determination function 113 (determination processing) and processing executed by the display control function 114 (display control processing) will be described. The determination function 113 determines a display mode based on the interest medical treatment information group, and the display control function 114 causes the terminal 50 used by the user to display the determined display mode on its display.

For example, the determination function 113 determines a screen layout for displaying the interest medical treatment information group (anti-cancer drug administration, ultrasound, tumor marker, . . . ) as a display mode, and the display control function 114 causes the terminal 50 used by the user to display the determined display mode on its display. As a specific example of the layout, the determination function 113 causes the terminal 50 to display the related nodes as a screen with the determined layout on its display in order, starting with a node closest to the axis of medical care, for example. Alternatively, the determination function 113 may cause the terminal 50 to display the related information on its display to be linked together, for example, from left to right on a single screen, or top to bottom displayed in scrolling, or may determine a display range by specifying the number of nodes.

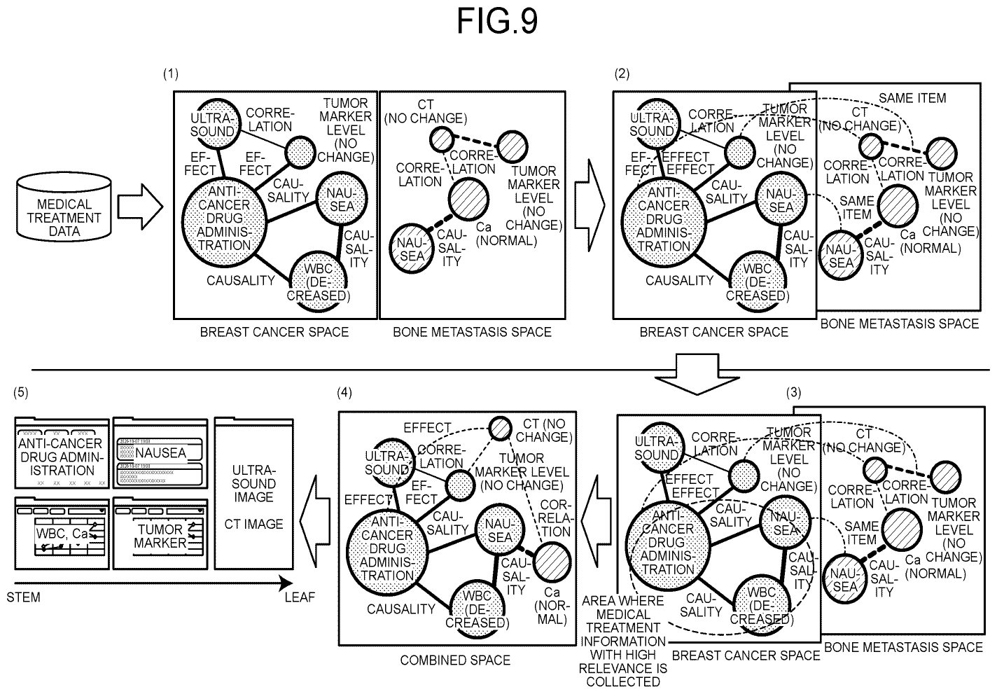

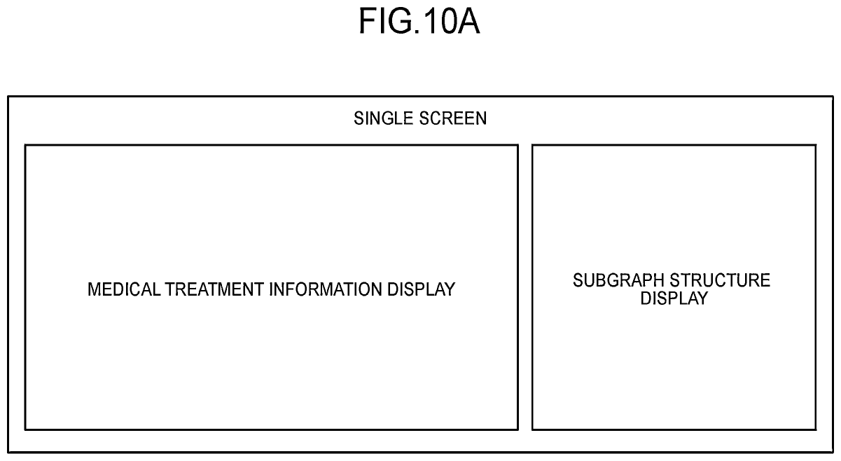

FIG. 8 is a diagram illustrating an example of the determination processing and the display control processing in the present embodiment. In the example illustrated in FIG. 8, the result of the anti-cancer drug administration and the result of a sample test on WBC and Ca are displayed on the left side of the screen as information corresponding to the nodes “anti-cancer drug administration”, “WBC (decreased)” and “Ca (normal)” on the combined medical treatment ontology. The left side of the screen is the start point of the axis of medical care to be displayed. A report on nausea and the result of a sample test on the tumor marker are displayed on the center of the screen as information corresponding to the related nodes “nausea” and “tumor marker level (no change)” associated with the above-described nodes “anti-cancer drug administration”, “WBC (decreased)”, and “Ca (normal)”. Ultrasound images and CT images are displayed on the right side of the screen as information corresponding to the related nodes “ultrasound” and “CT (no change)” associated with the above-described nodes “anti-cancer drug administration”, “WBC (decreased)”, and “Ca (normal)”. For example, in FIG. 8, it is indicated that nausea occurs due to anti-cancer drug administration but not due to hypercalcemia. Here, FIG. 9 is a diagram illustrating processing executed by the medical information processing apparatus 100 according to the present embodiment to summarize the processing illustrated in FIGS. 5A, 5B, 7, and 8.

The determination function 113 also determines a layout for displaying a subgraph structure corresponding to the interest medical treatment information group on the medical treatment ontology together with the interest medical treatment information group (anti-cancer drug administration, ultrasound, tumor marker, . . . ) as a display mode, and the display control function 114 causes the terminal 50 used by the user to display the determined display mode on its display.

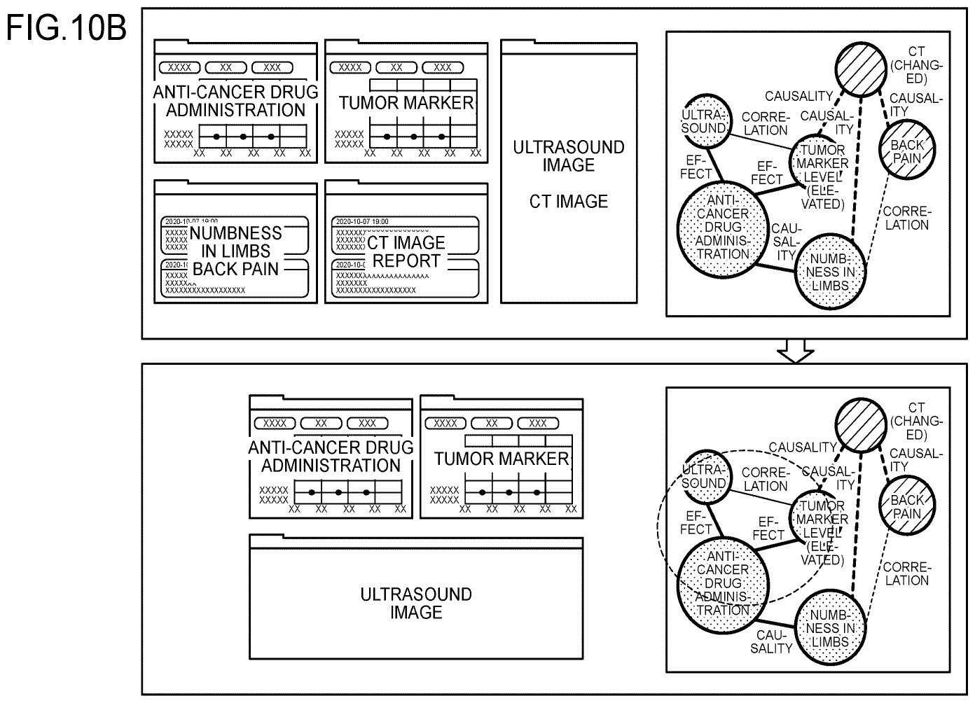

FIGS. 10A and 10B are diagrams illustrating another example of the determination processing and the display control processing in the present embodiment. It is indicated in FIG. 10A that a single screen displays the interest medical treatment information group (anti-cancer drug administration, ultrasound, tumor marker, . . . ) as the display of the medical treatment information, and a subgraph structure corresponding to the interest medical treatment information group on the combined medical treatment ontology as the display of the graph structure. In this case, information corresponding to the nodes “anti-cancer drug administration”, “ultrasound”, “tumor marker level (elevated)”, “numbness in limbs”, “CT (changed)”, and “back pain” on the combined medical treatment ontology is displayed on the screen, as illustrated in the upper figure in FIG. 10B. Specifically, the result of the anti-cancer drug administration, the result of a sample test related to the numbness in limbs and back pain, the result of a sample test related to the tumor marker, a report of CT images, CT images, and ultrasound images are displayed on the screen. Here, it is assumed that the nodes “anti-cancer drug administration”, “ultrasound”, and “tumor marker level (elevated)” on the screen are selected by the user, as illustrated on the right side of the lower figure in FIG. 10B. In this case, as illustrated on the left side of the lower figure in FIG. 10B, the result of the anti-cancer drug administration, the result of the sample test on the tumor marker, and the ultrasound images are displayed on the screen as information corresponding to the selected nodes “anti-cancer drug administration”, “ultrasound”, and “tumor marker level (elevated)”.

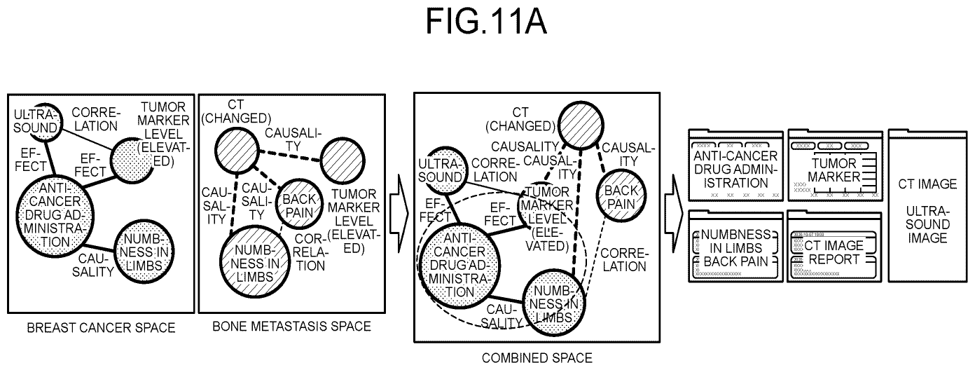

FIGS. 11A and 11B are diagrams illustrating yet another example of the determination processing and the display control processing in the present embodiment.

In the example illustrated in FIG. 11A, the determination function 113 determines, as a display mode, a layout focusing on breast cancer treatment provided that the risk of bone metastasis is low, with information on bone metastasis as reference information. The display control function 114 then causes the terminal 50 used by the user to display the determined display mode on its display.

In the example illustrated in FIG. 11B, the determination function 113 determines, as a display mode, a layout focusing on bone metastasis treatment provided that the risk of bone metastasis is high, with information on breast cancer as reference information. The display control function 114 then causes the terminal 50 used by the user to display the determined display mode on its display.

In the present embodiment, information on a plurality of diseases is displayed on a single screen to allow the user to understand the relationship between pieces of the medical treatment information.

In the present embodiment, the medical concepts may indicate not only concepts related to diseases, but also concepts related to treatment methods and phases of medical treatment. For example, different treatments are applied to the same disease, or different relationships are present between pieces of the medical treatment information as the phase of medical treatment progresses, which will also be taken into account. The medical concepts may also indicate concepts related to medical departments. For example, the combination of pieces of the medical treatment information appropriate for each department will also be taken into account because different departments have different views even on the basis of the same information depending on the type of treatment applied to a patient. The medical concepts may also indicate concepts related to physicians. For example, a medical treatment ontology can be generated for each physician, for example, to integrate the views on information from different specialist physicians, or to reflect the views on information from veteran physicians into the views of new physicians. The medical concepts may also indicate concepts related to patients. For example, symptoms and treatment methods (including factors such as fertility, amount, and time) can reflect patient values by increasing input values of nodes.

### Second Embodiment

In the first embodiment, the determination function 113 determines the screen layout for displaying the interest medical treatment information group as a display mode based on the interest medical treatment information group, and the display control function 114 causes the terminal 50 used by the user to display the determined display mode on its display. Here, provided that there is medical treatment information that some users already want to focus on, the users may prefer to determine a display layout based on the medical treatment information. Therefore, in a second embodiment, the terminal 50 displays an interest medical treatment information group that a user wants to focus on, with priority.

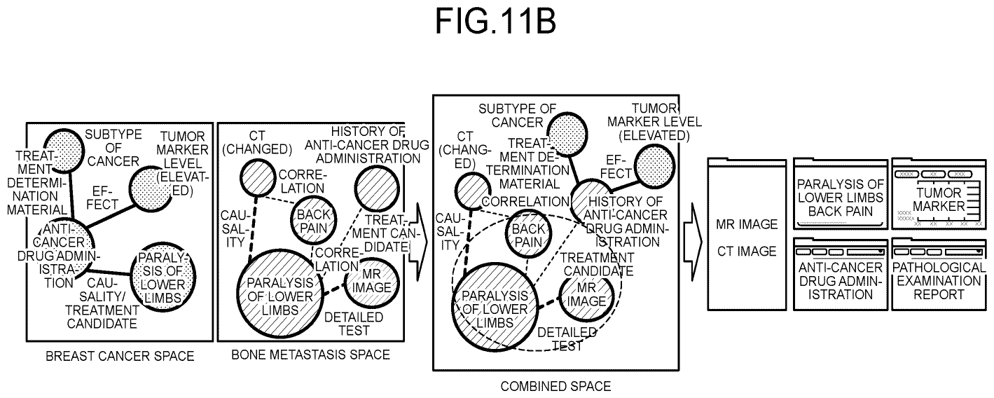

FIG. 12 is a diagram illustrating processing executed by the medical information processing apparatus 100 according to the second embodiment. In the example illustrated in FIG. 12, it is assumed that the nodes “anti-cancer drug administration”, “WBC (decreased)”, and “nausea” in the subgraph structures “breast cancer space” and “bone metastasis space” were selected by the user. In this case, the identification function 112 sets high input values for the nodes “anti-cancer drug administration”, “WBC (decreased)”, and “nausea”, preferentially employs the internal states of the nodes selected by the user, and identifies a subgraph structure including the nodes with the high internal states as an interest medical treatment information group. The determination function 113 then determines a screen layout for displaying the interest medical treatment information group as a display mode based on the interest medical treatment information group, and the display control function 114 causes the terminal 50 used by the user to display the determined display mode on its display.

In the present embodiment, the user can understand the relationship with other medical treatment information by taking the medical treatment information that the user wants to focus on into account.

### Third Embodiment

In a third embodiment, the granularity of a node in the medical treatment ontology may be changed to display medical treatment information with the appropriate granularity depending on the medical treatment scene. The definition of multiple layers with various granularities on the medical treatment ontology allows the granularity of the node to be changed. For example, the granularity may be changed by user designations or applications by the user selecting a patient on the screen, or may be changed according to the medical scene or patient's medical treatment information. Here, the identification function 112 sets higher input values for nodes in a high layer and lower input values for nodes in a low layer.

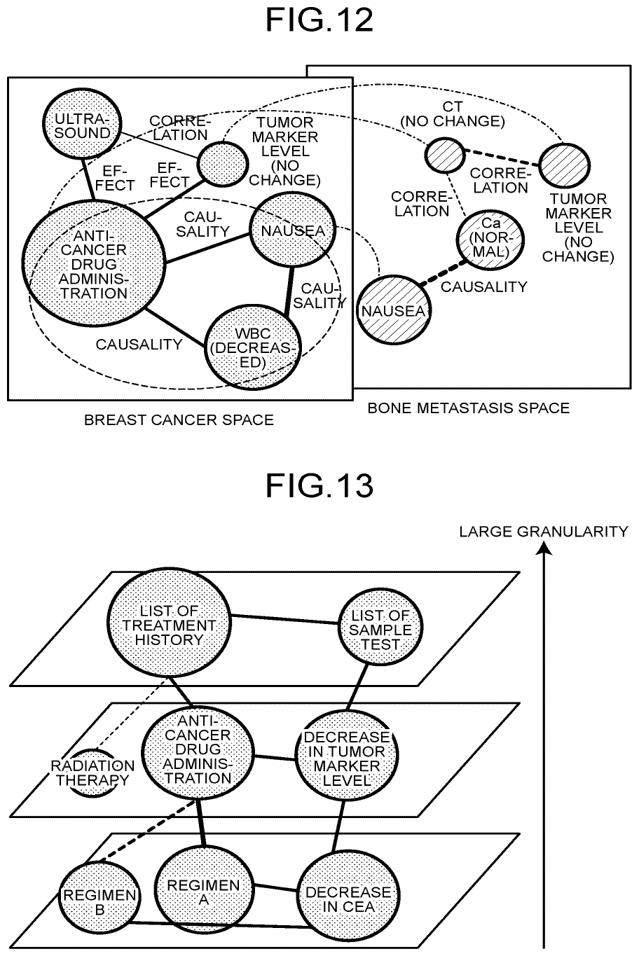

FIG. 13 is a diagram illustrating processing executed by the medical information processing apparatus 100 according to the third embodiment. In the example illustrated in FIG. 13, the granularities of the nodes “radiation therapy”, “anti-cancer drug administration”, and “decrease in tumor marker” are lower than the granularities of the nodes “treatment history” and “list of sample test” associated with the nodes and higher than the granularities of the nodes “regimen A”, “regimen B”, and “decrease in carcinoembryonic antigen (CEA)” associated with the nodes. For example, in a case in which the user wants to compare a plurality of patients, the display control function 114 displays medical treatment information by increasing the granularity according to an instruction from the user.

### Fourth Embodiment

In a fourth embodiment, medical treatment information necessary for user decision-making is displayed with priority. For example, in a case in which the user is referring to the medical treatment information on a screen, the identification function 112 sets a higher input value for a node corresponding to the medical treatment information that the user is referring to. In this way, the display control function 114 supports the user decision-making by prioritizing the display of the medical treatment information necessary for the user decision-making based on the information that the user refers to.

For example, even though a trained model that predicts diseases using machine learning is used, the medical treatment information necessary for the user decision-making can be preferentially displayed. For example, in a case in which the prediction result obtained by the trained model indicates a high probability of predicting a disease, the identification function 112 sets a higher input value for the node corresponding to the medical treatment information used by the trained model. In this way, the display control function 114 supports the user decision-making by prioritizing the display of the medical treatment information necessary for the user decision-making based on the disease prediction.

### Fifth Embodiment

In the first embodiment, the relationship between pieces of medical treatment information is considered based on the medical treatment information that the patient has. In a fifth embodiment, medical treatment information that is highly relevant to the axis of medical care is considered as missing information even though the patient does not have the medical treatment information, and the user is prompted to confirm the medical treatment information.

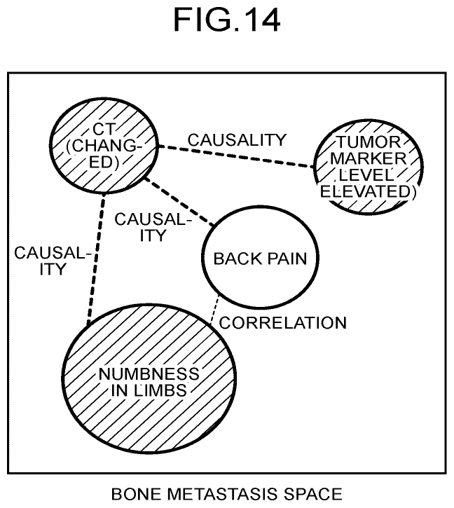

FIG. 14 is a diagram illustrating processing executed by the medical information processing apparatus 100 according to the fifth embodiment. In an example illustrated in FIG. 14, there is no medical treatment information on back pain in the patient's medical treatment information. Therefore, the identification function 112 identifies the node “back pain” as a node with a high internal state in the subgraph structure “bone metastasis space” based on its relationship with other nodes. In this case, the display control function 114 causes the terminal 50 to display a display mode based on the interest medical treatment information group and also causes the terminal 50 to display a message about back pain to prompt the user to confirm the information. Alternatively, the display control function 114 causes the terminal 50 to display a screen of the subgraph structure “bone metastasis space” illustrated in FIG. 14 regarding back pain and perform highlighting or other emphasis on the node “back pain” on the screen to prompt the user to confirm the information. In this way, the user can understand the relationship between pieces of the medical treatment information.

The individual components of the individual apparatuses illustrated in the present embodiment are functionally conceptual, and are not always required to be physically configured as illustrated in the figures. In other words, the specific forms of distribution and integration of the individual apparatuses are not limited to those illustrated in the figures, and all or some of the apparatuses can be configured in a functionally or physically distributed and integrated manner in any units depending on various types of loads or use conditions. Furthermore, all or some of the individual processing functions executed by the individual apparatuses can be implemented by CPU and computer programs analyzed and executed by the CPU, or can be implemented by hardware using a wired logic.

The method described in the present embodiment can also be implemented by executing a pre-prepared computer program on a computer such as a personal computer or workstation. This computer program can be distributed via the Internet or other networks. This computer program can also be recorded in a non-transitory computer readable medium, such as hard disk, flexible disk (FD), CD-ROM, MO, or DVD, and executed by being read from the medium by a computer.

According to at least one of the embodiments described above, the user can understand the relationship between pieces of the medical treatment information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising:
processing circuitry configured to acquire medical treatment information of a patient; and
storage circuitry configured to store a medical treatment ontology related to a medical concept, wherein
the processing circuitry
identifies an interest medical treatment information group on the medical treatment ontology based on information related to the medical treatment information, and
determines a display mode based on the interest medical treatment information group.

2. The medical information processing apparatus according to claim 1, wherein
the storage circuitry stores a plurality of the medical ontologies related to a plurality of the medical concepts, and
the processing circuitry
combines the medical ontologies based on the medical treatment information to generate a combined medical treatment ontology, and
identifies the interest medical treatment information group on the combined medical treatment ontology.

3. The medical information processing apparatus according to claim 1, wherein
the medical concept indicates a concept related to a disease or a treatment method.

4. The medical information processing apparatus according to claim 1, wherein
the medical treatment ontology is a graph structure in which medical concepts function as nodes, and a relationship between the medical concepts functions as an edge.

5. The medical information processing apparatus according to claim 4, wherein
the processing circuitry derives a relevance between individual nodes corresponding to the medical treatment information in the graph structure, and identifies a subgraph structure including a node with high relevance and a node adjacent to the node with high relevance as the interest medical treatment information group.

6. The medical information processing apparatus according to claim 1, wherein
the processing circuitry determines a layout for displaying, as the display mode, the interest medical treatment information group.

7. The medical information processing apparatus according to claim 1, wherein
the processing circuitry determines a layout for displaying, as the display mode, a subgraph structure corresponding to the interest medical treatment information group on the medical treatment ontology together with the interest medical treatment information group.

8. A medical information processing method comprising:
acquiring medical treatment information of a patient;
storing a medical treatment ontology related to a medical concept;
identifying an interest medical treatment information group on the medical treatment ontology based on information related to the medical treatment information; and
determining a display mode based on the interest medical treatment information group.

9. A non-transitory computer readable medium comprising instructions that cause a computer to execute:
acquiring medical treatment information of a patient;
storing a medical treatment ontology related to a medical concept;
identifying an interest medical treatment information group on the medical treatment ontology based on information related to the medical treatment information; and
determining a display mode based on the interest medical treatment information group.
